(19) United States
(12) Patent Application Publication
(10) Pub. No.: US 2009/0306903 A1
(43) Pub. Date: Dec. 10, 2009

(54) COMPOUND SCREENING METHOD AND APPARATUS

(75) Inventor: Nobuhiko Ogura, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJI FILM CORPORATION, Tokyo (JP)

(21) Appl. No.: 12/161,617

(22) PCT Filed: Jan. 16, 2007
(86) PCT No.: PCT/JP2007/050870

§ 371 Date: Feb. 11, 2009

(30) Foreign Application Priority Data
Jan. 19, 2006 (JP) 2006-010799

(51) Int. Cl. G01N 33/50; G06F 19/00

(52) U.S. Cl. 702/19

(57) ABSTRACT

A screening apparatus divides measurement data representing the amount of binding between one kind of protein and each of a plurality of kinds of compounds, obtained by a measurement apparatus, into groups, each including data obtained in a same measurement condition. The screening apparatus obtains a representative value of measurement data that is obtained when the protein and a compound are not bound to each other for each of the groups by using the measurement data belonging to the respective groups, and sets a threshold value for extracting a hit compound for each of the groups by using corrected measurement data, obtained by correcting the measurement data for each of the groups so that the representative value obtained for each of the groups becomes the same value. Then, a hit compound is extracted by comparing the threshold value with the corrected measurement data.

### TECHNICAL FIELD

The present invention relates to a compound screening method and apparatus. Particularly, the present invention relates to a compound screening method and apparatus for extracting a hit compound, which binds to one kind of protein, from a plurality of kinds of compounds. In the compound screening method and apparatus, the hit compound is extracted based on measurement data representing the amount of binding between the one kind of protein and each of the plurality of kinds of compounds.

### BACKGROUND ART

Generally, many kinds of compounds included in medicines achieve their effects and functions by chemically binding to protein in living bodies. Therefore, in development of a medicine, it is important to know whether a candidate compound for the medicine binds to protein. Ideally, a compound included in the medicine should be bindable only to a protein of interest, and it should not be bindable to the other proteins. That is because if the compound is also bindable to proteins other than the protein of interest, so-called adverse side-effects may occur. Therefore, in development of a medicine, screening is performed to extract a compound that is bindable only to a protein of interest from candidate compounds for the medicine.

Various kinds of apparatuses have been proposed to perform screening on candidate compounds for medicines. For example, a measurement apparatus is well known (please refer to “Journal of Spectroscopical Research of Japan”, Vol. 47, No. 1 (1998)). The measurement apparatus is an apparatus utilizing a phenomenon that when a surface plasmon is generated by total reflection of a light beam at the surface of a metal, an attenuated total-reflection angle changes based on a dielectric constant at the vicinity of the surface of the metal. The attenuated total-reflection angle is a specific reflection angle at which sharp attenuation (attenuated total reflection) of the intensity of light occurs in the totally-reflected light beam. Further, a similar measurement apparatus, for example, such as a leakage-mode measurement apparatus, which utilizes the attenuated total reflection, is also well known (please refer to “Journal of Spectroscopical Research of Japan”, Vol. 47, No. 1 (1998)).

As the apparatus using the principle of surface plasmon, “BIACORE3000”, manufactured by Biacore KK, or the like is well known, for example (please refer to “Real-Time Analysis Experiment Method of Interactions of Living-Body Substances”, Kazuhiro Nagata and Hiroshi Handa, published by Springer-Verlag Tokyo).

As a technique for performing compound screening by using the aforementioned apparatus, a technique for extracting a hit compound, which binds to one kind of protein, is being considered. In the technique, measurement data representing the amount of binding between the one kind of protein and each of a multiplicity of kinds of compounds, for example 10000 kinds of compounds, is obtained. Then, a hit compound is extracted, based on the measurement data, from the 10000 kinds of compounds.

In this technique, the hit compound is extracted based on a statistical processing result of 10000 kinds of measurement data obtained by measuring the amount of binding between the one kind of protein and each of the 10000 kinds of compounds. Specifically, for example, a threshold value for extracting the hit compound is set based on an average value of the values of the 10000 kinds of measurement data and the standard deviation of the values of the 10000 kinds of measurement data. Then, a compound corresponding to measurement data of which the value exceeds the threshold value is extracted as the hit compound.

Generally, in compound screening as described above, approximately 1% of screened compounds, namely 100 kinds of compounds out of 10000 kinds of compounds, are hit compounds. It needs two or three days or even longer period to perform screening on so many kinds of compounds.

However, if the measurement data that has been obtained as described above is widely dispersed, a compound that binds to a protein, but of which the amount of binding to the protein is small because of its low reaction speed or the like, is not detected in the dispersed data. Hence, such a compound is not extracted as a hit compound in some cases. Specifically, if the measurement data is widely dispersed, the value of measurement data obtained by using such a low-reaction compound becomes less than or equal to a threshold value for judging a hit compound in some cases. In such cases, the result of screening obtained by spending a plenty of time and expense is not sufficiently utilized. Hence, there are requests for more accurate screening of compounds to identify hit compounds.

### DISCLOSURE OF THE INVENTION

In view of the foregoing circumstances, it is an object of the present invention to provide a compound screening method and a compound screening apparatus that can improve the reliability of screening.

A first compound screening method of the present invention is a compound screening method for extracting, based on measurement data representing the amount of binding between one kind of protein and each of a plurality of kinds of compounds, a hit compound, which binds to the one kind of protein, from the plurality of kinds of compounds by obtaining the measurement data, the method comprising the steps of:

dividing the measurement data into groups, each including measurement data obtained in a same measurement condition;

obtaining a representative value of measurement data that is obtained when the protein and a compound are not bound to each other for each of the groups by using the measurement data belonging to the respective groups;

obtaining corrected measurement data by correcting the measurement data for each of the groups so that the representative value obtained for each of the groups becomes the same value;

setting a threshold value for extracting the hit compound by using the corrected measurement data; and

extracting the hit compound by comparing the threshold value with the value of the corrected measurement data.

A second compound screening method of the present invention is a compound screening method for extracting, based on measurement data representing the amount of binding between one kind of protein and each of a plurality of kinds of compounds, a hit compound, which binds to the one kind of protein, from the plurality of kinds of compounds by obtaining the measurement data, the method comprising the steps of:

dividing the measurement data into groups, each including data obtained in a same measurement condition;

setting a threshold value for extracting the hit compound for each of the groups by using measurement data belonging to the respective groups; and

extracting the hit compound by comparing the threshold value with the value of the measurement data in each of the groups.

The measurement condition may be a measurement date on which the amount of binding between each of the compounds and the protein is measured. Alternatively, the measurement condition may be a measurement sensor for measuring the amount of binding between each of the compounds and the protein.

If the compound screening method includes measurement channels for obtaining a plurality of kinds of measurement data based on measurement results obtained by measuring the amount of binding between each of the compounds and the protein in parallel, the measurement condition may be the measurement channels.

If the compound screening method is a method for measuring the amount of binding between each of the compounds and the protein by dissolving the respective compounds in a buffer solution, the measurement condition may be the production lot of the buffer solution.

If the compound screening method is a method for measuring the amount of binding between each of the compounds and the protein by using a plurality of measurement apparatuses, the measurement condition may be the measurement apparatuses.

The representative value may be obtained by using any kinds of methods as long as the value represents the value of measurement data when a protein and a compound do not bind to each other. For example, the representative value may be obtained by statistically processing measurement data belonging to each group.

The threshold value in the first compound screening method may be set based on a result of statistical processing of corrected measurement data, for example. Further, the threshold value in the second compound screening method may be set based on a result of statistical processing of measurement data in each group.

The measurement condition may be an ambient temperature during measurement for obtaining the measurement data.

The measurement data representing the amount of binding may be obtained by utilizing the principle of surface plasmon resonance. The measurement condition may be one of a measurement container (measurement vessel) used in measurement utilizing the principle of surface plasmon resonance, a flow path for measurement provided in the measurement container, an optical system for measuring an attenuated total-reflection angle, the optical system being separately provided for each flow path for measurement of the measurement container, elapsed time after preparation of a compound solution, the compound solution being prepared by dissolving each compound used for measurement, and the preparation lot of a refractive-index standard solution for correcting measurement utilizing the principle of surface plasmon resonance.

The measurement condition may be a combination of at least two of the measurement date, the measurement sensor, the measurement channels, the production lot of the buffer solution, the measurement apparatuses, an ambient temperature, the measurement container, the flow path for measurement, the optical system for measurement, the elapsed time after preparation of the compound solution and the preparation lot of the refractive-index standard solution.

The expression “so that the representative value obtained for each of the groups becomes the same value” refers not only to a case in which each of the representative values becomes completely the same value. For example, a difference between the largest representative value and the smallest representative value may be reduced, or a difference between any two of the representative values may be reduced. It is more desirable that each of the representative values is substantially the same value. Further, it is further more desirable that each of the representative values is exactly the same value.

A first compound screening apparatus of the present invention is a compound screening apparatus comprising:

a measurement unit for obtaining measurement data representing the amount of binding between one kind of protein and each of a plurality of kinds of compounds; and

a screening unit for extracting, based on the measurement data, a hit compound, which binds to the one kind of protein, from the plurality of kinds of compounds, wherein the screening unit includes a storage unit, a grouping unit, a representative value operation unit, a data correction unit, a threshold value setting unit and a hit compound extraction unit, and wherein the storage unit stores the measurement data obtained by the measurement unit, and wherein the grouping unit divides the measurement data stored in the storage unit into groups, each including measurement data obtained by the measurement unit in a same measurement condition, and wherein the representative value operation unit obtains a representative value of measurement data that is obtained when the protein and a compound are not bound to each other for each of the groups by using the measurement data belonging to the respective groups, and wherein the data correction unit obtains corrected measurement data by correcting the measurement data for each of the groups so that the representative value obtained for each of the groups becomes the same value, and wherein the threshold value setting unit sets a threshold value for extracting the hit compound by using the corrected measurement data, and wherein the hit compound extraction unit extracts the hit compound by comparing the threshold value with the value of the corrected measurement data.

A second compound screening apparatus of the present invention is a compound screening apparatus comprising:

a measurement unit for obtaining measurement data representing the amount of binding between one kind of protein and each of a plurality of kinds of compounds; and

a screening unit for extracting, based on the measurement data, a hit compound, which binds to the one kind of protein, from the plurality of kinds of compounds, wherein the screening unit includes a storage unit, a grouping unit, a threshold value setting unit and a hit compound extraction unit, and wherein the storage unit stores the measurement data obtained by the measurement unit, and wherein the grouping unit divides the measurement data stored in the storage unit into groups, each including measurement data obtained by the measurement unit in a same measurement condition, and wherein the threshold value setting unit sets a threshold value for extracting the hit compound in each of the groups by using measurement data belonging to the respective groups, and wherein the hit compound extraction unit extracts the hit compound by comparing the threshold value with the value of the measurement data in each of the groups.

We have reached the first screening method and apparatus of the present invention by focusing on the fact that most of a plurality of kinds of compounds does not bind to a protein, in other words, the number of hit compounds is small. We have also focused on the fact that the value of measurement data obtained when a protein and a compound are not bound to each other should be originally the same value regardless of the kind of the compound.

In the first screening method and apparatus of the present invention, measurement data representing the amount of binding between one kind of protein and each of a plurality of kinds of compounds is divided into groups, each including measurement data obtained in a same measurement condition, and a representative value, which represents the value of measurement data that is obtained when the protein and a compound are not bound to each other, is obtained for each of the groups by using the measurement data belonging to the respective groups. Further, corrected measurement data is obtained by correcting the measurement data for each of the groups so that the representative value obtained for each of the groups becomes the same value, and a threshold value for extracting a hit compound is set by using the corrected measurement data. Then, the hit compound is extracted by comparing the threshold value with the value of the corrected measurement data. Therefore, it is possible to further improve the reliability of screening.

Specifically, the value of the measurement data that is obtained when the protein and the compound are not bound to each other should be the same value regardless of the kind of the compound if measurement is accurately performed. However, the value of such measurement data is dispersed due to various factors. Hence, in a group of measurement data that has a similar tendency of error, the degree of dispersion of the values of the measurement data obtained when the protein and a compound are not bound to each other is less than that of dispersion of all of measurement data obtained when the protein and a compound are not bound to each other. The group of measurement data that has a similar tendency of error is a group of measurement data obtained in the same measurement condition.

Therefore, it is possible to obtain corrected measurement data that can more accurately represent the amount of binding between the protein and each compound by obtaining the representative value for each of the groups and by removing an error caused by a difference in the measurement condition by correcting the measurement data of each of the groups so that the representative value of each of the groups becomes the same value as the representative values of the other groups. Accordingly, it is possible to make the value of measurement data that is obtained when the protein and a compound are not bound to each other become the same value in all of the groups in a more accurate manner. Consequently, it is possible to obtain the corrected measurement data that can more accurately represent the amount of binding between the protein and each compound. The number of hit compounds included in the plurality of kinds of compounds is very small, and the number of hit compounds in each group of measurement data that is obtained in the same measurement condition is very small. Therefore, even if measurement data belonging to each group is used to obtain a representative value of measurement data that is obtained when the protein and a compound are not bound to each other, it is possible to obtain the representative value without being substantially affected by the measurement data of a hit compound or hit compounds.

Further, since the corrected measurement data is used, it is possible to set a threshold value that can enable more accurate extraction of a hit compound. For example, even if the reaction speed of a compound is slow and the amount of binding between the compound and the protein is small, the compound is still detected in the dispersed measurement data and extracted as a hit compound. Therefore, it is possible to further improve the reliability of screening.

In the second screening method and apparatus of the present invention, measurement data representing the amount of binding between one kind of protein and each of a plurality of kinds of compounds is divided into groups, each including measurement data obtained in a same measurement condition, and a threshold value for extracting the hit compound is set for each of the groups by using measurement data belonging to the respective groups. Further, a hit compound is extracted by comparing the threshold value for each of the groups with the value of the measurement data. Therefore, it is possible to further improve the reliability of screening.

Specifically, a threshold value in which an error caused by a difference in the measurement condition has been removed can be set as a threshold value for a group in which an error in the value of measurement data has a similar tendency. Here, the group in which an error in the value of measurement data has a similar tendency is a group of measurement data obtained in a same measurement condition. Therefore, compared with a conventional method, it is possible to more accurately extract a hit compound by extracting a hit compound from each of the groups by using the threshold value for each of the groups, in which the error caused by the difference in the measurement condition has been removed. For example, even if the reaction speed of a compound is slow and the amount of binding between the compound and the protein is small, the compound is still detected in the dispersed measurement data and extracted as a hit compound. Hence, it is possible to further improve the reliability of screening.

### BRIEF DESCRIPTION OF THE DRAWINGS

FIG. 1 is a schematic conceptual diagram illustrating the configuration of an apparatus for carrying out a compound screening method of the present invention;

FIG. 2 is an enlarged perspective view of a measurement container;

FIG. 3A is a diagram illustrating data obtained on the first day in a compound measurement data library;

FIG. 3B is a diagram illustrating data obtained on the second day in the compound measurement data library;

FIG. 3C is a diagram illustrating data obtained on the third day in the compound measurement data library;

FIG. 4A is a diagram showing a histogram created by using measurement data;

FIG. 4B is a diagram showing a histogram created by dividing the measurement data into groups, each including measurement data obtained in the same measurement condition;

FIG. 5 is a diagram showing a histogram created by using corrected measurement data, which is obtained by removing a variation in measurement according to each measurement date;

FIG. 6A is a diagram illustrating a difference in the temperature of a compound solution that flows through a flow path for measurement on the first day;

FIG. 6B is a diagram illustrating a difference in the temperature of a compound solution that flows through the flow path on the second day;

FIG. 7A is a diagram illustrating extraction of a hit compound from group 1 by setting a threshold value for each group;

FIG. 7B is a diagram illustrating extraction of a hit compound from group 2 by setting a threshold value for each group; and

FIG. 7C is a diagram illustrating extraction of a hit compound from group 3 by setting a threshold value for each group.

### BEST MODE FOR CARRYING OUT THE INVENTION

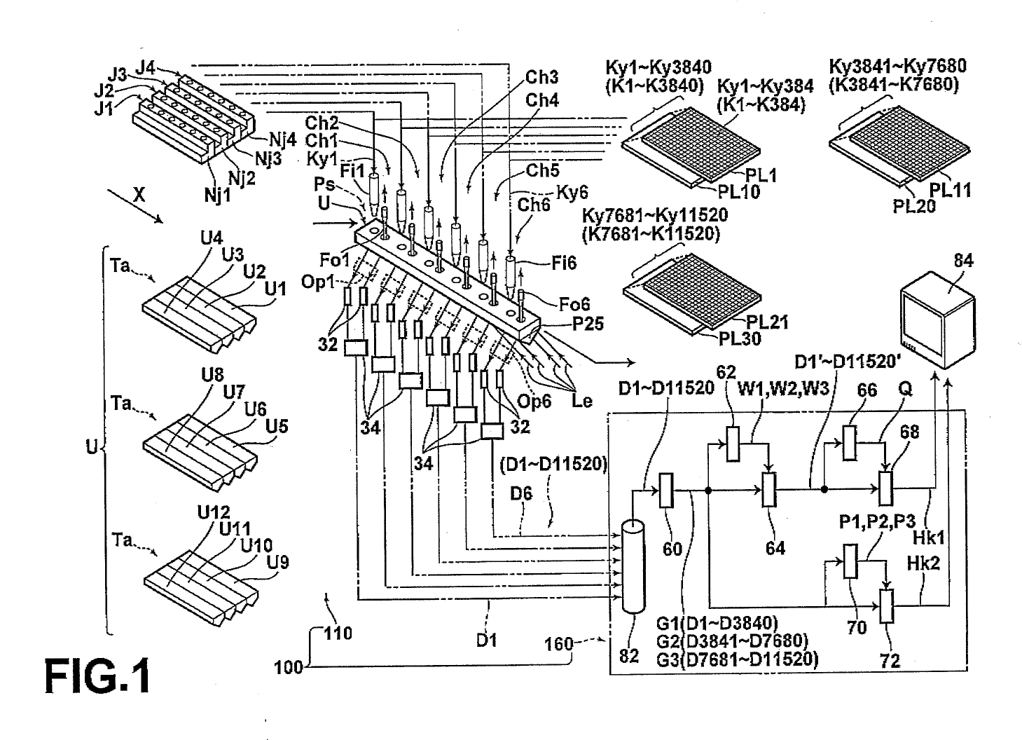

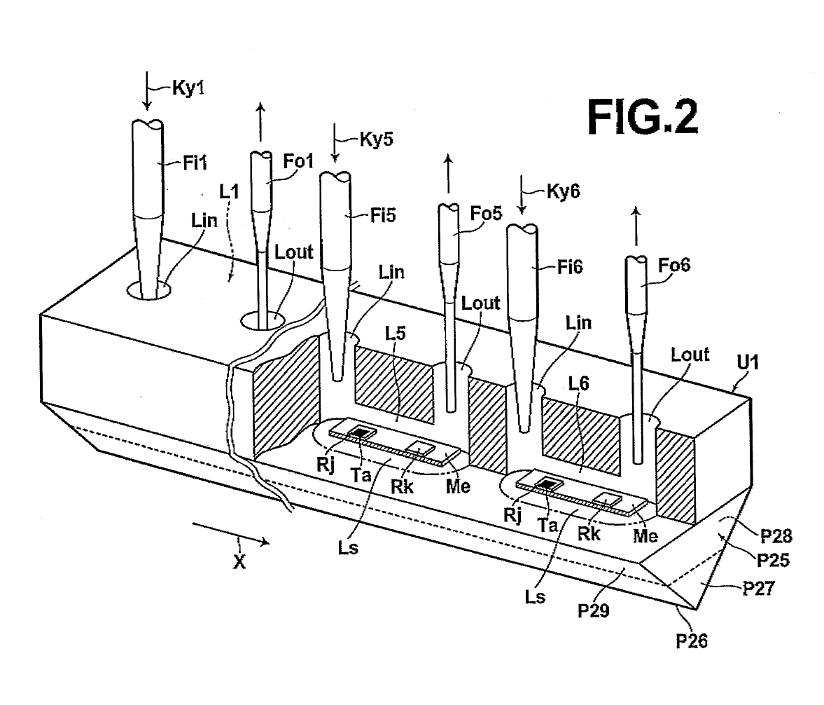

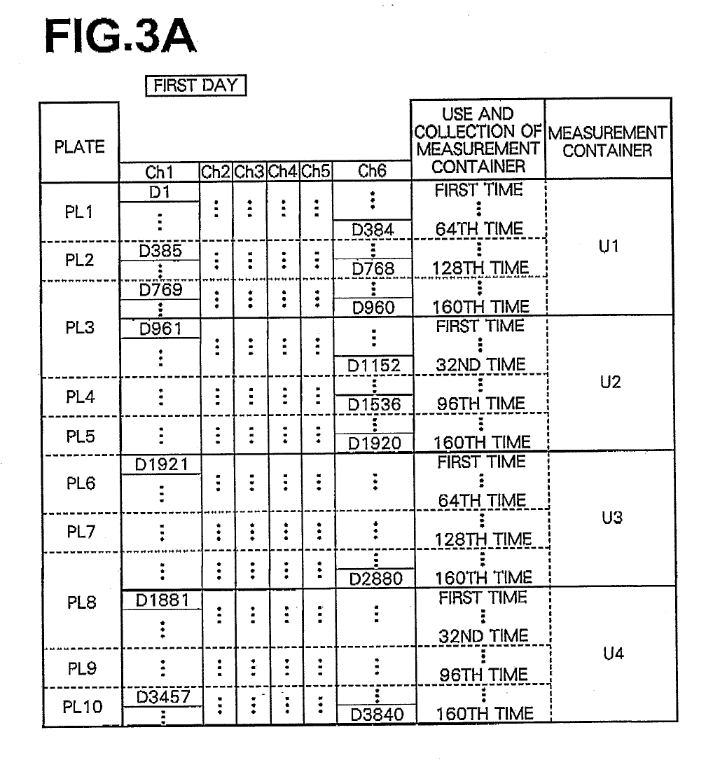

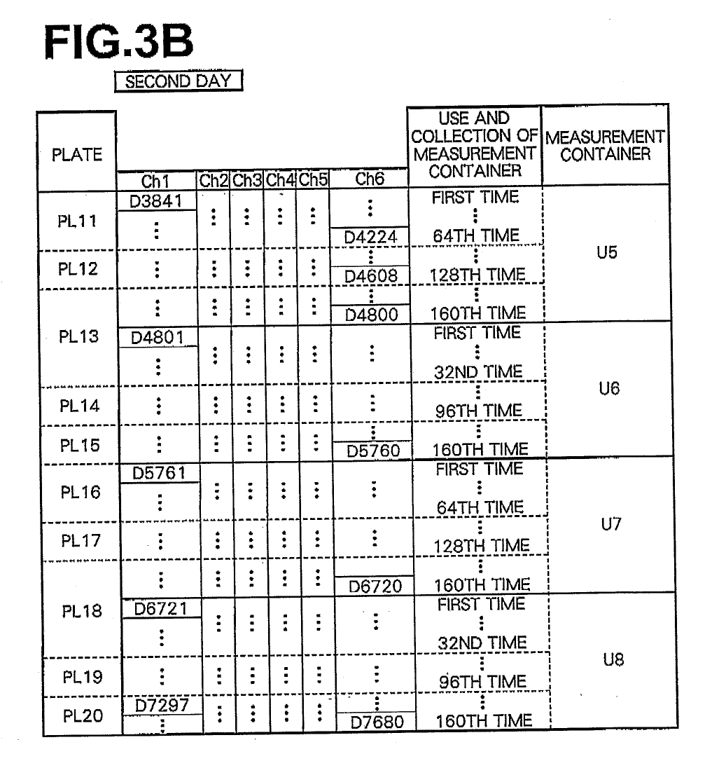

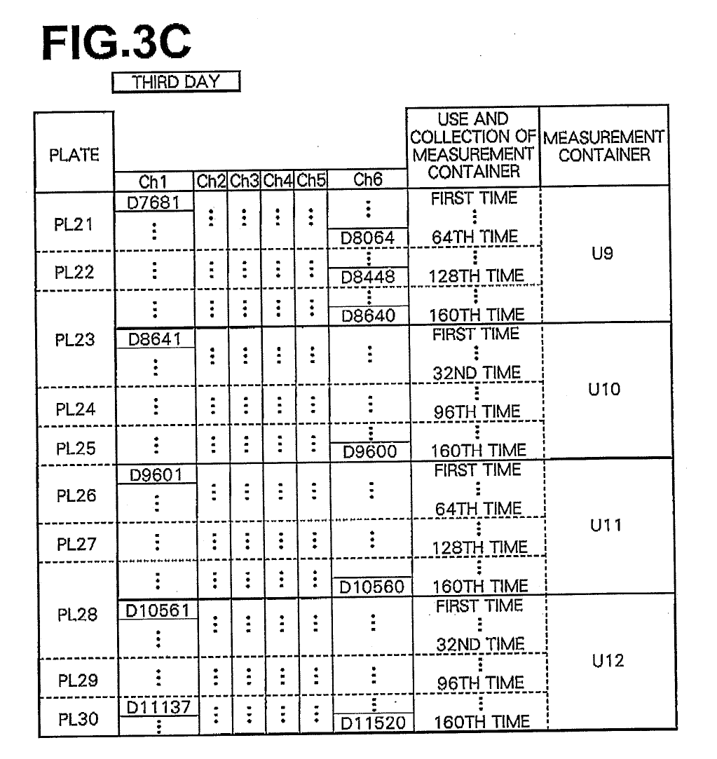

Hereinafter, embodiments of the present invention will be described with reference to the attached drawings. FIG. 1 is a schematic conceptual diagram illustrating the configuration of a compound screening apparatus for carrying out a compound screening method of the present invention. FIG. 2 is an enlarged perspective view of a measurement container (measurement vessel). FIGS. 3A, 3B and 3C are diagrams illustrating a compound measurement data library.

A compound screening apparatus 100, illustrated in FIG. 1, performs the compound screening method of the present invention. The compound screening apparatus 100 includes a measurement apparatus 110 and a screening apparatus 160. The measurement apparatus 110 is a measurement unit for obtaining each measurement data D1, D2, . . . , representing the amount of binding between one kind of protein Ta and each of a plurality of kinds of compounds K1, K2, . . . . The screening apparatus 160 is a screening unit for extracting a hit compound, which binds to the protein Ta, from the plurality of kinds of compounds K1, K2, . . . . The screening apparatus 160 extracts the hit compound based on the measurement data D1, D2, . . . .

The screening apparatus 160 includes a storage unit 82, a grouping unit 60, a representative value operation unit 62 and a data correction unit 64. The storage unit 82 stores measurement data D1, D2, . . . obtained at the measurement apparatus 110. The grouping unit 60 divides measurement data D1, D2, . . . stored in the storage unit 82 into groups G1, G2, . . . , each including measurement data obtained in the same measurement condition. The representative value operation unit 62 obtains representative values W1, W2, . . . of the values of measurement data obtained when a protein and a compound are not bound to each other for each of groups G1, G2, . . . , into which the measurement data has been divided by the grouping unit 60. The representative value operation unit 62 obtains representative values W1, W2, . . . using measurement data belonging to the respective groups. The data correction unit 64 obtains corrected measurement data D1′, D2′, . . . by correcting measurement data for each of groups G1, G2, . . . . The data correction unit 64 corrects the measurement data so that each of representative values W1, W2, . . . for groups G1, G2, . . . , obtained by the representative value operation unit 62, becomes the same value.

Further, the screening apparatus 160 includes a first threshold setting unit 66 and a first hit compound extraction unit 68. The first threshold setting unit 66 sets threshold value Q for extracting a hit compound by using corrected data D1′, D2′, . . . , obtained by the data correction unit 64. The first hit compound extraction unit 68 extracts hit compound Hk1 by comparing threshold value Q, obtained by the first threshold value setting unit 66, with each of the values of corrected measurement data D1′, D2′, . . . .

The screening apparatus 160 further includes a second threshold value setting unit 70 and a second hit compound extraction unit 72. The second threshold value setting unit 70 sets threshold values P1, P2, . . . for each of groups G1, G2, . . . , into which the measurement data has been divided by the grouping unit 60. Each of threshold values P1, P2, . . . is a threshold value for extracting a hit compound, and they are set by using measurement data belonging to the respective groups. The second hit compound extraction unit 72 extracts hit compound Hk2 from each of groups G1, G2, . . . by comparing each of threshold values P1, P2, . . . of the respective groups G1, G2, . . . with each of the values of measurement data belonging to the respective groups G1, G2, . . . .

Here, threshold value Q, set by the first threshold value setting unit 66, is a value based on the standard deviation of the values of corrected measurement data D1′, D2′, . . . .

Further, each of threshold values P1, P2, . . . , set by the second threshold value setting unit 70, is a value based on a standard deviation obtained for each of groups G1, G2, . . . . The standard deviation obtained for each of groups G1, G2, . . . is the standard deviation of the values of measurement data belonging to the respective groups G1, G2, . . . .

Next, a compound screening method carried out by the compound screening apparatus 100 will be described in detail. Here, a case in which a measurement date is adopted as the measurement condition will be described.

As illustrated in FIG. 1, the measurement apparatus 110 performs measurement using 12 measurement containers (measurement vessels) U1 through U12, plates PL1 through PL30 and standard solution containers Nj1 through Nj4. In each of measurement containers U1 through U12 (hereinafter, collectively referred to as measurement containers U), one kind of protein Ta has been immobilized. Plates PL1 through PL30 hold compound solutions Ky1, Ky2, . . . , each of which is obtained by dissolving one of compounds K1, K2, . . . (hereinafter, collectively referred to as compounds K) in a buffer solution. Compounds K1, K2, . . . are compounds that are different from each other. Standard solution containers Nj1 through Nj4 hold refractive-index standard solutions J1 through J4, respectively. The refractive-index standard solutions J1 through J4 have different refractive indices from each other (four kinds of refractive indices).

Each of measurement containers U1 through U12 is repeatedly used and discarded after being used 160 times.

Each of plates PL1 through PL30 holds 384 kinds of compound solutions, which are different from each other. Therefore, 11520 kinds of compound solutions are held on plates PL1 through PL30 in total.

The refractive indices of the refractive-index standard solutions J1 through J4, held in standard solution containers Nj1 through Nj4, are different from each other. The refractive index of each of the refractive-index standard solutions J1 through J4 has been obtained in advance by measurement or the like. Further, each of the refractive-index standard solutions J1 through J4 is prepared so that the distribution range of the refractive indices of the refractive-index standard solutions J1 through J4 includes the distribution range of the refractive indices of compound solutions Ky1, Ky2, . . . .

The refractive-index standard solutions J1 through J4 are mainly used to correct an error in measurement data caused by a difference in an attenuated total reflection angle in each flow path for measurement, which will be described later. The difference in the attenuated total-reflection angle is caused by a difference in the thickness of gold film Me provided in each of the flow paths for measurement, or the like. Measurement using each of the refractive index standard solutions J1 through J4 is performed once in every 160 times of repetitive measurement using each measurement container U.

On the first day, measurement is performed on compound solutions Ky1 through Ky3840 (3840 kinds of compound solutions in total), which are held on plates PL1 through PL10, by using measurement containers U1 through U4.

On the second day, measurement is performed on compound solutions Ky3841 through Ky7680 (3840 kinds of compound solutions in total), which are held on plates PL11 through PL20, by using measurement containers U5 through U8.

On the third day, measurement is performed on compound solutions Ky7681 through Ky11520 (3840 kinds of compound solutions in total), which are held on plates PL21 through PL30, by using measurement containers U9 through U12. Accordingly, experiments (tests or assays) concerning binding of each of the compounds to the one kind of protein end.

As described above, the measurement apparatus 110 measures the amount of binding between the one kind of protein and each of 11520 kinds of compounds in three days.

As illustrated in the enlarged perspective view of measurement container U1 in FIG. 2, each of measurement containers U has an elongate (narrow) shape extending in one direction (the direction of arrow X in FIG. 2). In each of measurement containers U, six flow paths L1 through L6 for measurement, which have the same shape, are provided along the longitudinal direction (the direction of arrow X in FIG. 2) of measurement container U1. The six flow paths L1 through L6 for measurement (hereinafter, collectively referred to as flow paths L for measurement) are paths through which compound solutions Ky1, Ky2, . . . flow. Each of compound solutions Ky1, Ky2, . . . is a solution obtained by dissolving the respective compounds K1, K2, . . . in a buffer solution. Compound solutions Ky flow through flow paths L for measurement in the direction of arrow X. Further, gold film Me has been deposited on a total reflection surface Ls by sputtering. The total reflection surface Ls is the bottom of each of the six flow paths L for measurement in each of measurement containers U. The total reflection surface Ls forms one of the surfaces of a prism P25, which has an elongate (narrow) shape that forms a part of measurement container U1. The prism P25 is formed in such a manner that a triangular shape extends in direction X. The total reflection surface Ls is a surface that totally reflects laser light Le, which will be described later. Further, the prism P25 may be formed by removing a triangular prism portion P27 extending in direction X, that includes a ridge (edge) P26 facing the total reflection surface Ls.

Further, linker layers are provided on gold film Me, which has been deposited on total reflection surface Ls of each of flow paths L for measurement. The same linker layers are provided at two positions of each of flow paths L for measurement, namely, at an upstream-side position and at a downstream-side position. Further, the one kind of protein Ta is immobilized on an upstream-side linker layer Rj. Meanwhile, nothing is immobilized on a downstream-side linker layer Rk. In measurement container U1, an inlet Lin for injecting each of compound solutions Ky is provided on the upstream side of each of flow paths L for measurement. An outlet Lout for discharging injected compound solutions Ky is provided on the downstream side of each of flow paths L for measurement. Each of measurement containers U1 through U12 has the same structure.

One of compound solutions Ky1 through Ky11520 is injected to each of flow paths L for measurement of each of the measurement containers. Compound solutions Ky1 through Ky11520 are compound solutions obtained by dissolving 11520 kinds of compounds K1 through K11520, respectively. Then, the amount of binding between each of compounds K and the protein Ta immobilized on the upstream-side linker layer Rj is measured. Neither the upstream-side linker layer Rj nor the downstream-side linker layer Rk binds to any one of compounds K1 through K11520.

The amount of binding is an immobilized amount of compound K with respect to the protein Ta on the upstream-side linker layer Rj. The immobilized amount corresponds to a change in mass per unit area on the surface of the upstream-side linker layer Rj, and the change in mass is caused by immobilization of compound K with respect to protein Ta.

The compound screening apparatus 100, which is structured as described above, obtains measurement data D1 through D11520. Measurement data D1 through D11520 is data representing the amount of binding between the one kind of protein Ta, immobilized in each of the flow paths for measurement, and each of the 11520 kinds of compounds K1 through K11520. Then, the compound screening apparatus 100 extracts, based on the values of the measurement data, a hit compound that binds to protein Ta from compounds K1 through K11520. The one kind of protein Ta has been immobilized in each of the six flow paths L1 through L6, which are provided for each of measurement containers U1 through U12 (72 flow paths for measurement in total).

Next, the action of the measurement apparatus 110 for measuring the amount of binding between the protein and each of the compounds will be described.

When measurement container U1 is conveyed and positioned at measurement position Ps, each of the formation area of the upstream-side linker layer Rj and the formation area of the downstream-side linker layer Rk on the total reflection surface Ls is irradiated with laser light Le through a prism surface P28 of the prism P25. The prism surface P28 is a surface that is different from the total reflection surface Ls. Irradiation with the laser light Le is performed so that the beam (light flux) of the laser light Le condenses at each of the formation area of the upstream-side linker layer Rj and the formation area of the downstream-side linker layer Rk.

The laser light Le is totally reflected by the formation area (hereinafter, referred to as an act area) of the upstream-side linker layer Rj and the formation area (hereinafter, referred to as a ref area) of the downstream-side linker layer Rk on the total reflection surface Ls. Then, the laser light Le is emitted to the outside in a divergent state through a prism surface P29 of the prism P25.

Each beam of the laser light Le is emitted to the outside in a divergent state, and enters each measurement sensor 32 including a two-dimensional CCD (charge-coupled device) or the like. Each of the measurement sensors 32 receives the beam of the laser light Le and measures the position of a dark line generated when the laser light Le is totally reflected. Specifically, an attenuated total-reflection angle caused by surface plasmon resonance is measured. Accordingly, an attenuated total-reflection angle at each of the act area and the ref area is obtained. The act area is an area in which protein Ta has been immobilized, and the ref area is an area in which protein Ta has not been immobilized. If a solution or liquid that does not react with the protein Ta flows through flow path L for measurement, the attenuated total-reflection angle in the act area and the attenuated total reflection in the ref area are substantially the same.

Further, a difference in angles obtained by subtracting a degree of change in an attenuated total-reflection angle in the ref area from a degree of change in an attenuated total-reflection angle in the act area when compound solution Ky flows through flow path L for measurement corresponds to the amount of binding between the protein Ta in the act area and the compound in the compound solution.

A binding-amount obtainment unit 34 continuously receives signals representing attenuated total-reflection angles in each of the act area and the ref area from the measurement sensor 32. The binding-amount obtainment unit 34 obtains measurement data representing the amount of binding by subtracting a degree of change in the attenuated total-reflection angle in the ref area from a degree of change in the attenuated total-reflection angle in the act area.

The value of the measurement data represents the amount of binding between protein Ta in the act area and the compound per unit area. The unit of the value of the measurement data is RU (resonant unit).

Regarding obtainment of the measurement data representing the amount of binding, please refer to “Real-Time Analysis Experiment Method of Interactions of Living-Body Substances”, Kazuhiro Nagata and Hiroshi Handa, published by Springer-Verlag Tokyo), or the like.

As described above, when measurement container U1 is positioned at measurement position Ps, a predetermined amount of compound solutions Ky1 through Ky6 held on plate PL1 is drawn by suction by injection pipes Fi1 through Fi6, respectively. The injection pipes Fi1 through Fi6 that have sucked the respective compound solutions Ky1 through Ky6 are transferred and inserted to inlets Lin of flow paths L1 through L6 for measurement, respectively. Each of compound solutions Ky1 through Ky6 is injected from injection pipes Fi1 through Fi6, and they flow through flow paths L1 through L6 for measurement, respectively. Then, measurement is performed to obtain measurement data D1 through D6, each representing the amount of binding between protein Ta and compounds K1 through K6, respectively.

The compound solution Ky injected to each of flow paths L1 through L6 for measurement can be discharged therefrom by suction by discharge pipes Fo1 through Fo6, which have been inserted to outlets Lout. As described above, the measurement apparatus 110 is structured so that the solutions that flow through flow paths L1 through L6 for measurement can be replaced. Therefore, a solution or liquid for preprocessing or post-processing of measurement of the amount of binding can flow through each of flow paths L1 through L6 for measurement.

At measurement position Ps, the act area and the ref area in each of flow paths L1 through L6 for measurement of measurement container U1 are separately irradiated with laser light Le. Then, each of 12 measurement sensors 32 measures an attenuated total-reflection angle. The measurement sensor 32 is separately provided for each of the act areas and the ref areas. Further, six binding-amount obtainment units 34 are provided for flow paths L1 through L6 for measurement, respectively. Each of the binding-amount obtainment units 34 obtains measurement data representing the amount of binding.

Here, measurement unit Ch1 for obtaining measurement data representing the amount of binding between a compound and a protein by using flow path L1 for measurement is defined as measurement channel 1. Measurement unit Ch2 for obtaining measurement data representing the amount of binding between a compound and a protein by using flow path L2 for measurement is defined as measurement channel 2, . . . measurement unit Ch6 for obtaining measurement data representing the amount of binding between a compound and a protein by using flow path L6 for measurement is defined as measurement channel 6.

When measurement on compound solutions Ky1 through Ky6 ends, recycling process is performed. The recycling process is a process for making the state of each of flow paths L1 through L6 of measurement container U1 return to a state thereof before the measurement has been performed. Specifically, a compound that has bound to the protein in the act area is removed by breaking the bond between the compound and the protein. Further, compound solutions Ky1 through Ky6, the buffer solution and the like that remain in the act area and the ref area or on the walls of flow paths L1 through L6 for measurement are removed by washing.

Then, measurement (second measurement) on compound solutions Ky7 through Ky12 is performed using the recycled measurement container U1.

As described above, recycling process is performed on measurement container U1 after every measurement. Measurement container U1 is repeatedly used 160 times, and measurement is performed on compound solutions Ky1 through Ky960. Accordingly, measurement data D1 through D960 corresponding to measurement of the respective compound solutions. Ky1 through Ky960 is obtained. Before the measurement using measurement container U1 is performed, data for correcting measurement on compound solutions Ky1 through Ky960 is obtained by making refractive-index standard solutions J1 through J4 flow through measurement container U1.

After measurement is performed by repeatedly using measurement container U1 160 times, measurement container U1 is transferred and removed from measurement position Ps, and next measurement container U2 is placed at measurement position Ps.

The measurement container U2 is repeatedly used 160 times for measurement in a manner similar to the aforementioned measurement. Measurement data D961 through D1920, each representing the amount of binding between each of compounds K961 through K1920 and protein Ta, is obtained by making compound solutions Ky961 through Ky1920 flow through flow paths L1 through L6 for measurement of measurement container U2.

Measurement is also performed using measurement containers U3 and U4 in a similar manner. Accordingly, measurement data D1 through D3840, each representing the amount of binding between each of compounds K1 through K3840 and protein Ta, is obtained by measurement using measurement containers U1 through U4, and measurement on the first day ends.

In measurement on the second day, measurement is performed using measurement containers U5 through U8 in a manner similar to the aforementioned measurement. Accordingly, measurement data D3841 through D7680, each representing the amount of binding between each of compounds K3841 through K7680 and protein Ta, is obtained.

In measurement on the third day, measurement is performed using measurement containers U9 through U12 in a manner similar to the aforementioned measurement. Accordingly, measurement data D7681 through D11520, each representing the amount of binding between each of compounds K7681 through K11520 and protein Ta, is obtained.

The measurement data D1 through D11520, obtained as described above, is input from the measurement apparatus 110 to the screening apparatus 160.

FIG. 3A is a diagram illustrating data obtained on the first day in a compound measurement data library. FIG. 3B is a diagram illustrating data obtained on the second day in the compound measurement data library. FIG. 3C is a diagram illustrating data obtained on the third day in the compound measurement data library. As illustrated in FIGS. 3A through 3C, measurement data D1 through D11520 obtained in three days can be organized as a compound measurement data library including a matrix of 640 rows×6 columns for each day. The matrices. may be arranged next to one another in the data library. Specifically, the compound measurement data library includes a matrix of 640 rows×6 columns, illustrated in FIG. 3A, representing measurement data D1 through D3840, obtained on the first day. The compound measurement data library also includes a matrix of 640 rows×6 columns, illustrated in FIG. 3B, representing measurement data D3841 through D7680, obtained on the second day. The compound measurement data library also includes a matrix of 640 rows×6 columns, illustrated in FIG. 3C, representing measurement data D7681 through D11520, obtained on the third day. Accordingly, measurement data D1 through D11520 can be organized and arranged in the compound measurement data library.

In each of the matrices, six columns correspond to measurement channels 1 through 6 or flow paths L1 through L6 for measurement. Each of 640 rows corresponds to six sets of measurement data that are simultaneously measured using each measurement container U.

Further, measurement data D1 through D960 is obtained by repeatedly using measurement container U1 160 times. In other words, measurement data D1 through D960 is obtained in the first through 160th measurement using measurement container U1. Similarly, measurement data D961 through D1920 is obtained by repeatedly using measurement container U2 160 times, and measurement data D1921 through D2880 is obtained by repeatedly using measurement container U3 160 times.

Then, at the end of the third day, measurement data D10561 through D11520 is obtained by 160 times of measurement using measurement container U12.

Further, measurement data D1 through D384 is obtained by performing measurement on 384 kinds of compound solutions Ky1 through Ky384 held on plate PL1. Then, measurement data D385 through D768 is obtained by performing measurement on 384 kinds of compound solutions Ky385 through Ky768 held on plate PL2. Then, measurement data D769 through D1536 is obtained by performing measurement on 384 kinds of compound solutions Ky769 through Ky1536 held on plate PL3.

Then, at the end of the third day, measurement data D11137 through D11520 is obtained by performing measurement on 384 kinds of compound solutions Ky11137 through Ky11520 held on plate PL30.

As described above, the measurement container used for measurement is switched from measurement container U1 to measurement container U2 during measurement of 384 kinds of compound solutions Ky769 through Ky1536 held on plate PL3.

Here, the measurement date of a group of measurement data D1 through D3840, obtained on the first day, is the same. The measurement date of a group of measurement data D3841 through D7680, obtained on the second day, is the same. Further, the measurement date of a group of measurement data D7681 through D11520, obtained on the third day, is the same, and the measurement date is one of measurement conditions.

It is expected that most of measurement data D1 through D11520 will be obtained in a state in which the protein and a compound are not bound to each other. Further, it is expected that measurement data obtained in a state in which the protein and a compound are bound to each other will be approximately 1% of the whole measurement data, or less than or equal to 2% of the whole measurement data at most.

Next, the action of the screening apparatus 160 for further clearly distinguishing measurement data obtained in a state in which the protein and a compound are not bound to each other from measurement data obtained in a state in which the protein and a compound are bound to each other will be described.

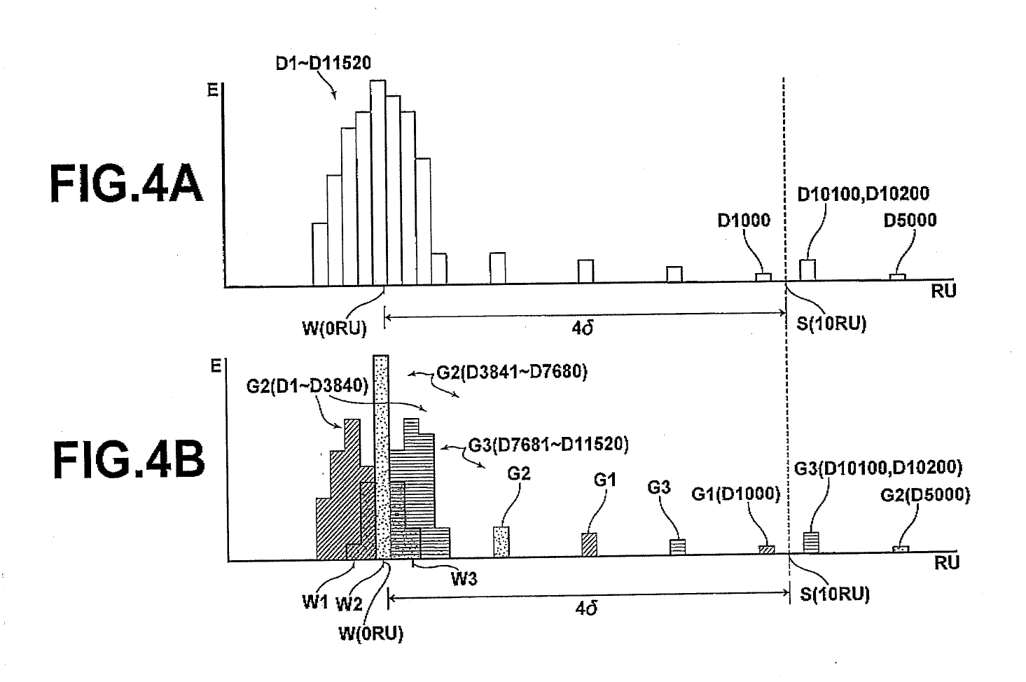

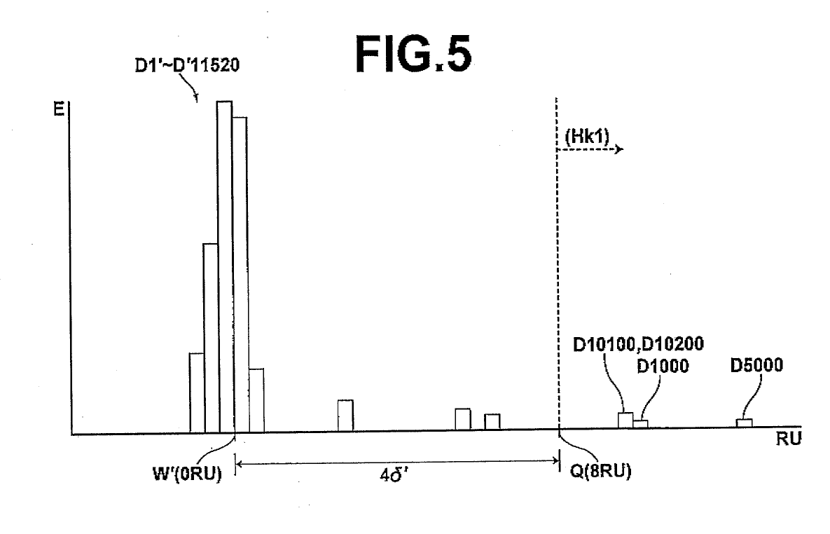

FIG. 4A is a diagram showing a histogram created by using the 11520 sets of measurement data. In the coordinates of FIG. 4A, the vertical axis represents frequencies (E) and the horizontal axis represents amounts of binding (RU). FIG. 4B is a diagram showing a histogram created by dividing the measurement data into groups, each including data obtained in the same measurement condition. FIG. 5 is a diagram showing a histogram created by using corrected measurement data, which is obtained by removing a variation (fluctuation) in measurement according to measurement dates. In the coordinates of FIG. 5, the vertical axis represents frequencies (E) and the horizontal axis represents amounts of binding (RU).

As illustrated in FIG. 4A, most of measurement data represents that the protein and a compound do not bind to each other. Therefore, most of the measurement data is present in the vicinity of 0RU. Meanwhile, measurement data representing that the protein and a compound bind to each other should be present in an area very far from 0RU on the positive side of 0RU, and only a small number of sets of such measurement data should be present.

The value of measurement data obtained in a state in which the protein and a compound are not bound to each other should be originally the same as the value of 0RU. However, the value of measurement data obtained in such a state is dispersed because measurement conditions, such as measurement dates for example, are different from each other, or the like. Therefore, the values of some measurement data obtained in the state in which the protein and a compound are not bound to each other are shifted to the negative side or to the positive side of 0RU.

In the screening apparatus 160, which has received measurement data D1 through D11520, the grouping unit 60 divides measurement data D1 through D11520 into groups G1, G2 and G3, each including measurement data obtained on the same measurement date. Specifically, as illustrated in FIG. 4B, measurement data D1 through D3840, obtained in measurement on the first day, is classified as group G1. Measurement data D3841 through D7680, obtained in measurement on the second day, is classified as group G2. Measurement data D7681 through D11520, obtained in measurement on the third day, is classified as group G3.

Further, a lookup table showing a correspondence between groups and measurement data, or the like is stored in the grouping unit 60 in advance. The grouping unit 60 divides the measurement data into groups based on the correspondence.

When measurement data D1 through D11520 is divided into groups G1, G2 and G3, it is recognized that group 1 includes many sets of measurement data, of which the values are less than 0RU. It is also recognized that group 2 includes many sets of measurement data, of which the values are in the vicinity of 0RU. Further, it is recognized that group 3 includes many sets of measurement data, of which the values are greater than 0RU.

Next, the representative value operation unit 62 obtains representative values W1, W2 and W3 of measurement data for groups G1, G2 and G3, respectively. The representative values W1, W2 and W3 are values when the protein and a compound are not bound to each other. The representative value operation unit 62 obtains the representative values W1, W2 and W3 using measurement data in the respective groups G1, G2 and G3.

When the representative value operation unit 62 obtains representative value W1 for group G1, first, the representative value operation unit 62 obtains average value W1′ and standard deviation σ1′ by using all of measurement data D1 through D3840 belonging to group G1. Then, the representative value operation unit 62 extracts measurement data of which the value is within a range obtained by adding the product of the standard deviation multiplied by 4 to the average value. In other words, measurement data, of which the value is less than or equal to W1′+(4×σ1′), is extracted. Then, an average value of the extracted measurement data is adopted as the representative value W1.

All of the measurement data, of which the value is less than or equal to the value obtained by adding the product of the standard deviation multiplied by 4 to the average value may not be measurement data obtained when the protein and a compound are not bound to each other. However, it can be expected that even if measurement data obtained when the protein and a compound are bound to each other is included, only a very small number of sets of such measurement data will be included. Therefore, it can be expected that the value of the representative value will be only slightly affected by such measurement data. Hence, the influence of such measurement data can be substantially ignored in screening.

Representative value W2 for group G2 and representative value W3 for group G3 are obtained in a similar manner.

Next, the data correction unit 64 corrects measurement data for each of groups G1, G2 and G3. The data correction unit 64 corrects the measurement data so that representative values W1, W2 and W3, obtained for groups G1, G2 and G3 by the representative value operation unit 62, become the same value. Here, the data correction unit 64 corrects the measurement data for each of groups G1, G2 and G3 so that representative values W1 and W3 become the same as representative value W2 (here, W2=0RU), for example. Specifically, each of the values of measurement data belonging to group G1 is shifted to the positive side. Each of the values of measurement data belonging to group G3 is shifted to the negative side. Each of the values of measurement data belonging group G2 is shifted by a shift amount of 0. Then, corrected measurement data D1′ through D3840′, D3841′ through D7680′ and D7681′ through D11520′ is obtained for groups G1, G2 and G3, respectively.

As illustrated in FIG. 5, the dispersion of whole corrected measurement data D1′ through D11520′, including corrected data D1′ through D3840′, D3841′ through D7680′ and D7681′ through D11520′, is reduced. Specifically, a variation in measurement according to each measurement date is removed, and the dispersion of corrected data D1′ through D11520′ is reduced.

Next, the first threshold-value setting unit 66 sets threshold value Q for extracting a hit compound by using corrected measurement data D1′ through D11520′, obtained by the data correction unit 64.

Here, threshold value Q (Q=W′+(4×σ′)) is obtained using average value W′ of corrected measurement data D1′ through D11520′ and standard deviation σ′ of corrected measurement data D1′ through D11520′. In FIG. 5, average value W′ (W′=0RU) is illustrated.

Finally, the first hit compound extraction unit 68 compares threshold value Q with the values of corrected measurement data D1′ through D11520′. Then, the first hit compound extraction unit 68 extracts a compound corresponding to corrected measurement data, of which the value exceeds threshold value Q, from the corrected measurement data D1′ through D11520′ as hit compound Hk1. The extracted hit compound Hk1 is displayed on a display device 84.

Here, corrected measurement data D1′ through D11520′ is data in which a variation in measurement according to each measurement is removed, and the dispersion of the data is small. Therefore, corrected measurement data D1′ through D11520′ can more accurately represent the amount of binding between a protein and a compound. Since corrected measurement data D1′ through D11520′ is used, it is possible to more accurately extract a hit compound than a case of extracting a hit compound using measurement data D1 through D11520, which has not been corrected.

Specifically, in the conventional method, an average value of measurement data D1 through D11520 is W and the standard deviation of measurement data D1 through D11520 is σ, as illustrated in FIGS. 4A and 4B. If a hit compound is extracted by using measurement data D1 through D11520, measurement data D10100, D10200, D5000 and the like, which exceed threshold value S (S=W+4σ), are extracted as hit compounds. However, measurement data D1000 or the like, which is less than or equal to threshold value S, is not extracted as a hit compound.

As illustrated in FIGS. 4A and 4B, average value W is 0RU (W=0RU), and threshold value S is 10RU (S=10RU).

In contrast, in the method according to the present invention, standard deviation σ′ of corrected measurement data D1′ through D11520′ is less than standard deviation σ of measurement data D1 through D11520, as illustrated in FIG. 5. Therefore, if a hit compound is extracted from corrected measurement data D1′ through D11520′ by using threshold value Q (Q=W′+4σ′), measurement data D1000 as well as measurement data D10100, D10200 and D5000 is extracted as a hit compound. In this case, the value of threshold value Q is 8RU (Q=8RU), as illustrated in FIG. 5.

In the method according to the present invention, when the representative value for each group is obtained, measurement data of a compound that has probably bound to a protein is removed. However, it is not necessary that such measurement data is removed. For example, since the number of compounds that bind to the protein is very small, an average value for each of the groups may be obtained by using all of measurement data belonging to the respective groups. Then, the average value may be adopted as a representative value of measurement data obtained when the protein are not bound to a compound. Even if such average value is adopted as the representative value, it is possible to substantially achieve an effect similar to the aforementioned method. Specifically, it is possible to clearly distinguish measurement data obtained when the protein and a compound are not bound to each other from measurement data obtained when the protein and a compound are bound to each other.

In the above description, correction of measurement using refractive-index standard solutions J1 through J4 was not described. However, measurement may be corrected by the binding-amount obtainment unit 34, for example.

Specifically, before 160 times of repetitive measurement is performed using each measurement container U, refractive-index standard solutions J1 through J4 are injected to each of the flow paths for measurement instead of compound solutions, and measurement data for correction is obtained. Then, the measurement data for correction is input to the binding-amount obtainment unit 34. The refractive index of each of refractive-index standard solutions J1 through J4 is known. Therefore, the binding-amount obtainment unit 34 can correct, based on the measurement data for correction, each measurement data obtained by repeatedly performing measurement 160 times. The binding-amount obtainment unit 34 can correct each measurement based on a relationship between the refractive index (known value) of each of the solutions and an RU value (the value of measurement data for correction) obtained when each of the solutions flows through each of the flow paths for measurement.

Next, a variation in measurement data according to measurement dates will be described in detail.

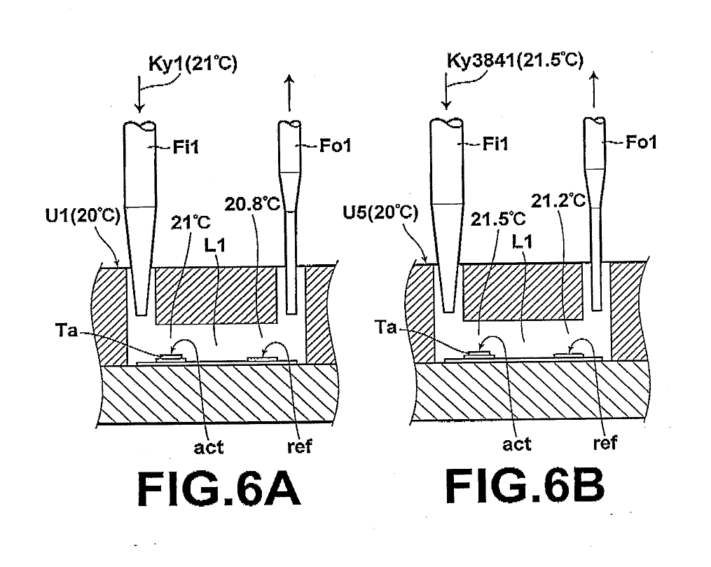

FIG. 6A is a diagram illustrating the distribution of the temperature of compound solution that flows through a flow path for measurement on the first day. FIG. 6B is a diagram illustrating the distribution of the temperature of compound solution that flows through the flow path on the second day.

If a difference in temperature according to measurement dates is large, a difference between the temperature of the compound solution and that of the measurement container varies according to measurement dates. As illustrated in FIG. 6A, in measurement on the first day, the temperature of compound solution Ky1 held on plate PL1 is 21° C., and the temperature of measurement container U1 is 20° C. The temperature of compound solution Ky1 injected to the act area of flow path L1 for measurement in measurement container U1 by injection pipe Fi1 is 21° C. In such a case, the heat of the compound solution Ky1 is absorbed by the measurement container U1 as the compound solution Ky1 flows through flow path L1 for measurement, and the temperature of compound solution Ky1 drops. Here, it is assumed that the temperature of compound solution Ky1 becomes 20.8° C. at the ref area.

In contrast, as illustrated in FIG. 6B, in the measurement on the second day, the temperature of compound solution Ky3841 held on plate PL11 is 21.5° C., and the temperature of measurement container U5 is 20° C. The temperature of compound solution Ky3841 injected to the act area of flow path L1 for measurement in measurement container U5 by injection pipe Fi1 is 21.5° C. In such a case, the heat of the compound solution Ky3841 is absorbed by the measurement container U5 as compound solution Ky3841 flows through flow path L1 for measurement, and the temperature of compound solution Ky3841 drops. Here, it is assumed that the temperature of compound solution Ky3841 becomes 21.2° C. at the ref area. Since the difference between the temperature of the compound solution and that of the measurement container on the second day is larger than the difference therebetween on the first day, the drop in the temperature of the compound solution on the second day is larger than the drop in the temperature on the first day.

Specifically, on the first day, a difference between the temperature of compound solution Ky1 at the act area and the temperature of compound solution Ky1 at the ref area is 0.2° C. In contrast, on the second day, the difference between the temperature of compound solution Ky3841 at the act area and that of compound solution Ky3841 at the ref area is 0.3° C. The refractive index or the like of each of the compound solutions changes based on the difference in the temperature, and the attenuated total-reflection angle also fluctuates. Therefore, even if the apparatus is structured in such a manner that reference is provided to compensate an error in measurement, as described above, it is impossible to compensate the fluctuation in the attenuated total-reflection angle, and the measurement data fluctuates.

Next, a case in which screening is performed by the screening apparatus 160 by using a method that is different from the aforementioned method will be described.

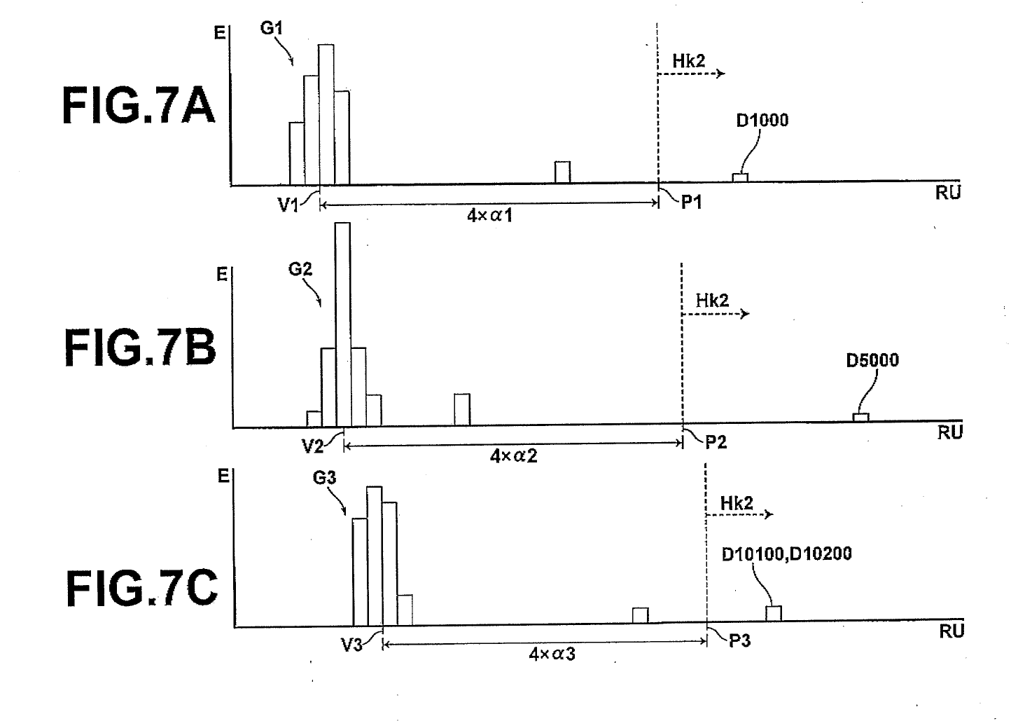

FIGS. 7A, 7B and 7C are diagrams illustrating cases in which a hit compound is separately extracted from each group by setting a threshold value for each of the groups. The vertical axis of the coordinates represents frequencies (E), and the horizontal axis represents amounts of binding (RU). FIG. 7A is a diagram illustrating a case of extracting a hit compound from group 1. FIG. 7B is a diagram illustrating a case of extracting a hit compound from group 2. FIG. 7C is a diagram illustrating a case of extracting a hit compound from group 3.

The second threshold-value setting unit 70 of the screening apparatus 160 sets each of threshold values P1, P2 and P3 for extracting a hit compound for each of groups G1, G2 and G3, into which the measurement data has been divided by the grouping unit 60. The second threshold-value setting unit 70 sets threshold values P1, P2 and P3 by using measurement data D1 through D3840, measurement data D3841 through D7680 and measurement data D7681 through D11520, respectively.

As illustrated in FIGS. 7A, 7B and 7C, threshold value P1 for group G1 is set based on average value Vi of measurement data D1 through D3840 in group G1 and standard deviation α1 of measurement data D1 through D3840 in group G1 (P1=V1+(4×α1)). Similarly, threshold value P2 for group G2 is set based on average value V2 of measurement data D3841 through D7680 in group G2 and standard deviation α2 of measurement data D3841 through D7680 in group G2 (P2=V2+(4×α2)). Further, threshold value P3 for group G3 is set based on average value V3 of measurement data D7681 through D11520 in group G3 and standard deviation α3 of measurement data D7681 through D11520 in group G3 (P3=V3+(4×α3)).

Next, the second hit compound extraction unit 72 extracts hit compound Hk2 from each of groups G1, G2 and G3. The second hit compound extraction unit 72 extracts hit compound Hk2 from group GI by comparing threshold value P1 with measurement data D1 through D3840. The second hit compound extraction unit 72 extracts a hit compound Hk2 from group G2 by comparing threshold P2 with measurement data D3841 through D7680. The second hit compound extraction unit 72 extracts hit compound Hk2 from group G3 by comparing threshold P3 with measurement data D7681 through D11520.

Specifically, as illustrated in FIG. 7A, measurement data D1000, of which the value is greater than or equal to threshold value P1, is extracted as hit compound Hk2 from group 1. Further, as illustrated in FIG. 7B, measurement data D5000, of which the value is greater than or equal to threshold value P2, is extracted as hit compound Hk2 from Group 2. Further, as illustrated in FIG. 7C, measurement data D10100 and D10200, of which the values are greater than or equal to threshold value P3, is extracted as hit compounds Hk2 from Group 3.

Alternatively, as the measurement condition, a measurement sensor for measuring the amount of binding between a compound and a protein or the like may be adopted instead of the measurement date. Further, if the compound screening method includes measurement channels for simultaneously obtaining a plurality of sets of measurement data by measuring the amount of binding between a compound and a protein in parallel, the measurement condition may be the measurement channel. Alternatively, if the compound screening method is a method for measuring the amount of binding between a compound and a protein by dissolving the compound in a buffer solution, the measurement condition may be the production lot of the buffer solution. Further, if the compound screening method is a method for measuring the amount of binding between a compound and a protein using a plurality of measurement apparatuses, the measurement condition may be the measurement apparatus.

Alternatively, a measurement condition, such as a measurement container, flow paths for measurement, an optical system for measurement, an ambient temperature during measurement, elapsed time after preparation of a compound solution (a plate for holding the compound solution), the preparation lot of a refractive-index standard solution or the like may be adopted instead of the aforementioned measurement conditions. The flow paths for measurement are a plurality of flow paths for measurement provided in a measurement container. Further, the optical system for measurement is an optical system for measuring an attenuated total-reflection angle, and the optical system for measurement is provided for each of the flow paths for measurement in the measurement container.

Next, each of the measurement conditions will be described.

### Regarding Measurement Container

The measurement container, which is repeatedly used for measurement, includes a narrow prism 25P (please refer to FIG. 2). An upstream-side linker layer Rj and a downstream-side linker layer Rk (hereinafter, collectively referred to as linker layers R) are arranged on gold film Me deposited on the total reflection surface Ls of the prism 25P. Each of the linker layers R is a layer formed by depositing an SAM layer (Self-Assembled Monolayer) and a CMD layer (carboxymethyldextran layer) on the gold film Me in this order. The SAM layer and the CMD layer are deposited on gold film Me by separately immersing (soaking or dipping) each prism P25 in a solution or liquid. Then, one kind of protein is immobilized on the linker layer R. Therefore, the performance of immobilization of the protein on the linker layer R is different in each of the measurement containers, which have been separately immersed in the solution. Therefore, if the measurement data is divided into groups, each including measurement data obtained using the same measurement container, which is a measurement condition, it is possible to further improve the reliability of screening.

### Regarding Plurality of Flow Paths for Measurement Provided in Measurement Container

Gold film Me is deposited on total reflection surface Ls of narrow prism P25, which forms the measurement container, by sputtering. Deposition of metal (gold) by sputtering is performed by placing prism P25 in a vacuum chamber in such a manner that total reflection surface Ls of prism P25 faces a sputtering target. The thickness of the gold film deposited by sputtering is slightly different at each position of total reflection surface Ls based on a difference in a distance between the sputtering target and gold film Me or the like. Further, the attenuated total-reflection angle that is measured by irradiating total reflection surface Ls with light changes depending on the thickness of the gold film. Further, a characteristic error (difference) in an attenuated total-reflection angle obtained by measurement is generated in each of the flow paths for measurement in the measurement container. The characteristic error in the attenuated total-reflection angle obtained by measurement has a tendency unique to each of the flow paths for measurement. Therefore, if the measurement data is divided into groups, each including measurement data obtained in the same flow path for measurement, which is a measurement condition, it is possible to improve the reliability of screening.

### Regarding Optical System for Measurement

When an attenuated total-reflection angle is measured, optical systems Op (Op1 through Op6, please refer to FIG. 1) for measurement transmit light through optical paths that are different from each other. The optical systems Op are provided for the flow paths for measurement of the measurement container, respectively. In each of the optical systems Op1 through Op6 for measurement, optical parts forming each of the optical paths and the arrangement thereof are slightly different from each other. Therefore, a characteristic error in an attenuated total-reflection angle obtained by measurement is generated. The characteristic error in the attenuated total-reflection angle has a unique tendency. Hence, if the measurement data is divided into groups, each including measurement data obtained using the same optical system, which is a measurement condition, it is possible to improve the reliability of screening.

### Regarding Ambient Temperature

A difference between the temperature of a compound solution held on a plate and the temperature of a measurement container is caused by an ambient temperature during measurement. A difference between the temperature of the compound solution passing the act area and that of the compound solution passing the ref area is caused by the difference between the temperature of the compound solution and that of the measurement container, as described above. Further, the value of the difference in temperature fluctuates, and the value of the refractive index of the compound solution fluctuates as the temperature fluctuates. Therefore, a characteristic error in an attenuated total-reflection angle obtained by measurement is caused by the difference in the ambient temperature. The characteristic error in the attenuated total-reflection angle obtained by measurement has a unique tendency. Hence, if the measurement data is divided into groups, each including measurement data obtained at the same ambient temperature, which is a measurement condition, it is possible to further improve the reliability of screening.

### Regarding Elapsed Time After Preparation of Compound Solution

### (Plate for Holding Compound Solutions)

The compound solution is prepared by dissolving a compound in a PBS solution (phosphate buffered solution). The compound is supplied by being mixed in undiluted 100% DMSO solution (Dimethyl Sulfoxide solution). The DMSO solution containing the compound is dissolved in the PBS solution, and the compound solution is obtained. Here, since the PBS solution evaporates as time elapses, the density of the compound solution changes, and the refractive index of the compound solution changes. Therefore, a characteristic error in an attenuated total-reflection angle obtained by measurement is generated according to a difference in elapsed time after preparation of the compound solution. The characteristic error in the attenuated total-reflection angle obtained by measurement has a unique tendency. Hence, if the measurement data is divided into groups, each including measurement data obtained at the same elapsed time after preparation of the compound solution, which is a measurement condition, it is possible to further improve the reliability of screening.

Further, as described above, PBS solution sucked by 384 injection pipes is simultaneously ejected to a plate for holding 384 kinds of compound solutions, and compound solutions are prepared. Therefore, elapsed time after preparation of each of the compound solutions is the same for all of the compound solutions on the same plate. Hence, the measurement condition may be the plate for holding a plurality of kinds of compound solutions. The elapsed time after preparation of the compound solution is elapsed time while the PBS solution is set in an evaporatable condition. Therefore, if the compound solution is sealed to prohibit evaporation of the PBS solution after preparation the compound solution, a time period in which evaporation is prohibit is not included in the elapsed time.

### Regarding Preparation Lot of Refractive-Index Standard Solution

The refractive-index standard solution is used to correct an error in measurement data caused by a difference in the attenuated total-reflection angle in each of the flow paths for measurement. As described above, the difference in the attenuated total-reflection angle is caused by a difference in the thickness of gold film Me for each of the flow paths for measurement. A plurality of kinds of refractive-index standard solutions, each having a different refractive index from each other, is prepared.

The refractive-index standard solution is prepared so that the refractive index thereof becomes a value in the vicinity of the refractive index of the compound solution. For example, if the compound solution is prepared so that the ratio between the compound supplied by being mixed with 100% DMSO solution and the PBS solution is 1:9, a refractive-index standard solution prepared by mixing the DMSO solution in the PBS solution approximately at 1:9 is used. More specifically, four kinds of refractive-index standard solutions, each having a different refractive index from each other, are prepared. The four kinds of refractive-index standard solutions are a solution prepared by mixing the DMSO solution with the PBS solution at 9.5:90.5, a solution prepared by mixing the DMSO solution with the PBS solution at 10.0:90.0, a solution prepared by mixing the DMSO solution with the PBS solution at 10.5:89.5, and a solution prepared by mixing the DMSO solution with the PBS solution at 11.0:89.0. These refractive-index standard solutions are used.

However, a slight error is generated in the ratio between the DMSO solution and the PBS solution at each preparation of the solution. Therefore, the refractive index of each of the refractive-index standard solutions is not completely the same as a target value, and an error is generated. Therefore, when correction is performed, a refractive-index standard solution that has a different refractive index from each other is used according to a difference in the lot of the refractive-index standard solution when the refractive-index standard solution was prepared. Consequently, a characteristic error in correction is generated. In other words, an error having a unique tendency is generated.

Therefore, if the measurement data is divided into groups, each including measurement data obtained using a refractive-index standard solution of the same preparation lot, which is a measurement condition, it is possible to further improve the reliability of screening. Specifically, if the measurement data is divided into groups, each including measurement data obtained by performing correction using a refractive-index standard solution of which the preparation lot is the same, it is possible to include measurement data that has the same tendency of measurement error in each of the groups. Hence, it is possible to further improve the reliability of screening.

The measurement condition may be any one of a measurement date, a measurement sensor, measurement channels, the production lot of a buffer solution, a measurement apparatus, a measurement container, a flow path for measurement, an optical system for measurement, an ambient temperature, elapsed time after preparation of the compound solution, the plate for holding the compound solution and the preparation lot of a refractive-index standard solution. Alternatively, the measurement condition may be a combination of at least two of the aforementioned measurement conditions.

Further, it is not necessary that the representative value is an average value of the measurement data. The value of measurement data, of which the frequency of appearance is the highest or the like, may be adopted as the representative value.

Further, it is not necessary that the threshold value is set based on the standard deviation of the values of measurement data. The threshold value may be set by any kinds of methods.

Further, it is not necessary that gold film Me is deposited on total reflection surface Ls. A metal film made of metal other than gold may be deposited on total reflection surface Ls. Further, it is not necessary that the gold film or the metal film is deposited by sputtering. The gold film or the metal film may be deposited by vacuum vapor deposition or the like.

Further, in the aforementioned embodiment, the compound screening method has been described by using a case in which measurement data representing the amount of binding between a protein and a compound is obtained by compensating external disturbance using reference. However, the compound screening method of the present invention may also be adopted when the measurement data is obtained without using reference.

The compound screening method may be adopted in compound screening for extracting a hit compound that binds to one kind of protein from a plurality of kinds of compounds, such as a compound screening performed using a leakage-mode measurement apparatus, for example. The compound screening method may also be adopted in compound screening using an apparatus that utilizes other kinds of measurement principle.

1-11. (canceled)

12. A compound screening method for extracting, based on measurement data representing the amount of binding between one kind of protein and each of a plurality of kinds of compounds, a hit compound, which binds to the one kind of protein, from the plurality of kinds of compounds, the method comprising the steps of:
obtaining the measurement data;
dividing the measurement data into groups, each including measurement data obtained in a same measurement condition;
obtaining a representative value of measurement data that is obtained when the protein and a compound are not bound to each other for each of the groups by using the measurement data belonging to the respective groups;
obtaining corrected measurement data by correcting the measurement data for each of the groups so that the representative value obtained for each of the groups becomes the same;
setting a threshold value for extracting the hit compound by using the corrected measurement data; and
extracting the hit compound by comparing the threshold value with the value of the corrected measurement data.

13. A compound screening method for extracting, based on measurement data representing the amount of binding between one kind of protein and each of a plurality of kinds of compounds, a hit compound, which binds to the one kind of protein, from the plurality of kinds of compounds, the method comprising the steps of:
obtaining the measurement data
dividing the measurement data into groups, each including measurement data obtained in a same measurement condition;
setting a threshold value for extracting the hit compound for each of the groups by using measurement data belonging to the respective groups; and
extracting the hit compound by comparing the threshold value with the value of the measurement data in each of the groups.

14. A compound screening method, as defined in claim 12, wherein the measurement condition is a measurement date on which the amount of binding between each of the compounds and the protein is measured.

15. A compound screening method, as defined in claim 13, wherein the measurement condition is a measurement date on which the amount of binding between each of the compounds and the protein is measured.

16. A compound screening method, as defined in claim 12, wherein the measurement condition is a measurement sensor for measuring the amount of binding between each of the compounds and the protein.

17. A compound screening method, as defined in claim 13, wherein the measurement condition is a measurement sensor for measuring the amount of binding between each of the compounds and the protein.

18. A compound screening method, as defined in claim 12, the method comprising:
measurement channels for obtaining a plurality of kinds of measurement data based on measurement results obtained by measuring the amount of binding between each of the compounds and the protein in parallel, wherein the measurement condition is the measurement channels.

19. A compound screening method, as defined in claim 13, the method comprising:
measurement channels for obtaining a plurality of kinds of measurement data based on measurement results obtained by measuring the amount of binding between each of the compounds and the protein in parallel, wherein the measurement condition is the measurement channels.

20. A compound screening method, as defined in claim 12, wherein the amount of binding between each of the compounds and the protein is measured by dissolving the respective compounds in a buffer solution, and wherein the measurement condition is the production lot of the buffer solution.

21. A compound screening method, as defined in claim 13, wherein the amount of binding between each of the compounds and the protein is measured by dissolving the respective compounds in a buffer solution, and wherein the measurement condition is the production lot of the buffer solution.

22. A compound screening method, as defined in claim 12, wherein the amount of binding between each of the compounds and the protein is measured by using a plurality of measurement apparatuses, and wherein the measurement condition is the measurement apparatuses

23. A compound screening method, as defined in claim 13, wherein the amount of binding between each of the compounds and the protein is measured by using a plurality of measurement apparatuses, and wherein the measurement condition is the measurement apparatuses.

24. A compound screening method, as defined in claim 12, wherein the measurement condition is an ambient temperature during measurement for obtaining the measurement data.

25. A compound screening method, as defined in claim 13, wherein the measurement condition is an ambient temperature during measurement for obtaining the measurement data.

26. A compound screening method, as defined in claim 12, wherein the measurement data representing the amount of binding is obtained by measurement utilizing the principle of surface plasmon resonance, and wherein the measurement condition is one of a measurement container used in measurement, a flow path for measurement provided in the measurement container, an optical system for measuring an attenuated total-reflection angle, the optical system being separately provided for each flow path for measurement in the measurement container, elapsed time after preparation of a compound solution for measurement, the solution being prepared by dissolving each of the compounds used for measurement, and the preparation lot of a refractive-index standard solution used for correction of the measurement.

27. A compound screening method, as defined in claim 13, wherein the measurement data representing the amount of binding is obtained by measurement utilizing the principle of surface plasmon resonance, and wherein the measurement condition is one of a measurement container used in measurement, a flow path for measurement provided in the measurement container, an optical system for measuring an attenuated total-reflection angle, the optical system being separately provided for each flow path for measurement in the measurement container, elapsed time after preparation of a compound solution for measurement, the solution being prepared by dissolving each of the compounds used for measurement, and the preparation lot of a refractive-index standard solution used for correction of the measurement.

28. A compound screening apparatus comprising:
a measurement unit for obtaining measurement data representing the amount of binding between one kind of protein and each of a plurality of kinds of compounds; and
a screening unit for extracting, based on the measurement data, a hit compound, which binds to the one kind of protein, from the plurality of kinds of compounds, wherein the screening unit includes a storage unit, a grouping unit, a representative value operation unit, a data correction unit, a threshold value setting unit and a hit compound extraction unit, and wherein the storage unit stores the measurement data obtained by the measurement unit, and wherein the grouping unit divides the measurement data stored in the storage unit into groups, each including measurement data obtained by the measurement unit in a same measurement condition, and wherein the representative value operation unit obtains a representative value of measurement data that is obtained when the protein and a compound are not bound to each other for each of the groups by using the measurement data belonging to the respective groups, and wherein the data correction unit obtains corrected measurement data by correcting the measurement data for each of the groups so that the representative value obtained for each of the groups becomes the same value, and wherein the threshold value setting unit sets a threshold value for extracting the hit compound by using the corrected measurement data, and wherein the hit compound extraction unit extracts the hit compound by comparing the threshold value with the value of the corrected measurement data.

29. A compound screening apparatus comprising:
a measurement unit for obtaining measurement data representing the amount of binding between one kind of protein and each of a plurality of kinds of compounds; and
a screening unit for extracting, based on the measurement data, a hit compound, which binds to the one kind of protein, from the plurality of kinds of compounds, wherein the screening unit includes a storage unit, a grouping unit, a threshold value setting unit and a hit compound extraction unit, and wherein the storage unit stores the measurement data obtained by the measurement unit, and wherein the grouping unit divides the measurement data stored in the storage unit into groups, each including measurement data obtained by the measurement unit in a same measurement condition, and wherein the threshold value setting unit sets a threshold value for extracting the hit compound for each of the groups by using measurement data belonging to the respective groups, and wherein the hit compound extraction unit extracts the hit compound by comparing the threshold value with the value of the measurement data in each of the groups.
